(19) United States
(12) Patent Application Publication
(10) Pub. No.: US 2009/0232785 A1
(43) Pub. Date: Sep. 17, 2009

(54) Cosmetic and/or dermatological composition for prevention and/or treatment of sensitive or dry skin

(75) Inventors: Lionel Breton, Versailles (FR); Roland Jourdain, Meudon La Foret (FR); Audrey Gueniche, Rueil Malmaison (FR); Isabelle Bureau-Franz, Morges (CH); Mathilde Fleith, La Tour-De Peilz (CH); Armand Malnoe, Epalinges (CH)

Correspondence Address: OLIFF & BERRIDGE, PLC, P.O. BOX 320850, ALEXANDRIA, VA, 22320-4850, US

(73) Assignees: L'Oreal, Paris (FR); NESTEC S.A., VEVEY (CH)

(21) Appl. No.: 11/989,694

(22) PCT Filed: Jul. 31, 2006
(86) PCT No.: PCT/FR2006/050768

§ 371 Date: Jul. 14, 2008

(30) Foreign Application Priority Data
Aug. 1, 2005 (FR) 0552411

(51) Int. Cl. A61K 35/74; A61K 8/99; A61Q 19/00; A61P 17/00

(52) U.S. Cl. 424/93.44; 424/93.4; 424/93.51; 424/93.5; 424/93.45; 424/93.46; 424/93.462

(57) ABSTRACT

The invention relates to a cosmetic and/or dermatological topical composition, of particular use for the prevention al and/or treatment of sensitive and/or dry skin, comprising an effective amount of at least one particularly probiotic microorganism and/or a fraction or metabolite thereof in combination with an effective amount of at least one polyunsaturated fatty acid and/or polyunsaturated fatty acid ester and/or a salt or derivative thereof in a physiologically-acceptable vehicle.

The present invention essentially relates to a topical composition, notably cosmetic and/or dermatological, intended more particularly for the prevention and the treatment of skin qualified as “sensitive and/or dry skin”.

Generally, the sensitive skin is defined by a particular reactivity of the skin.

This cutaneous reactivity classically results in the display of discomfort signs in response to the contact of the subject with a triggering member which can have various origins. It can be the application of a cosmetic product on the surface of the sensitive skin, food consumption, exposure to sharp variations in temperatures, air pollution and/or ultraviolet or infra-red rays. There are also associated factors such as the age and type of skin. Thereby, sensitive skin is more frequent among dry or fatty skin than among normal skin.

The appearance of these discomfort signs, which appear in the minutes following the contact of the subject with the triggering member, is one of the essential characteristics of sensitive skin. They are mainly dysesthesic sensations. “Dysesthesic sensations” means essentially more or less painful sensations felt in a cutaneous area such as tingling, formications, itching or pruritus, burns, heating, discomfort, tugging, etc. These subjective signs generally exist without visible chemical signs such as red spots and exfoliations. It is currently known that these irritation and cutaneous intolerance reactions are notably related to a release of neuropeptides by the nerve endings of the epidermis and dermis.

As opposed to the skin qualified as allergic, the reactivity of a sensitive skin does not result from an immunologic process, i.e. does not only occur on already sensitized skin, in response to the presence of an allergen. Its response mechanism is known as “aspecific”. For this reason, it needs to be distinguished from the skin showing inflammatory and allergic reactions of dermatosis, eczema, and/or ichthyosis type, and regarding which a certain number of treatments have already been proposed.

Thereby, WO 02/28402 describes that probiotic microorganisms can have a beneficial effect in the regulation of cutaneous over-sensitive reactions such as inflammatory and allergic reactions which result from an immunologic process as opposed to the reactivity of a sensitive skin. It is also reported in “Probiotics in the management of atopic eczema, Clinical and Experimental Allergy 2000”, Volume 30, pages 1604-1610, a study concerning the effect of probiotics on the infantile immune system mechanisms such as for example the atopic dermatitis. U.S. Pat. No. 5,756,088 describes a diet having prophylactic and therapeutic effects on the animal dermatosis. This diet comprises the ingestion of compositions comprising a polyunsaturated fatty acid and/or biotin, a Bifidobacterium, a bacterium with lactic acid or a Bacillus. As for WO 01/17365, it describes a method enabling the animal skin and fur to be improved by providing them with a nutritional agent comprising a prebiotic or probiotic agent.

With regards to document WO 01/45721, it proposes cosmetic, pharmaceutical, veterinary compositions with topical application, more particularly intended to prevent and/or reduce the disorders induced by the pathogenics of the cutaneous system. For this, these compositions take advantage of the capability of certain lactic bacteria to adhere, on the one hand, to the cutaneous cells and on the other hand, to regulate the attachment of cutaneous pathogenics.

U.S. Pat. No. 5,656,268 offers biological products associating lactic ferments with a vegetable oil.

In fact, none of these documents are concerned with the prevention and/or treatment of skin qualified as sensitive, which we notably find in the adult, particularly when this sensitive skin is associated with dry skin. The dry skin primarily appears with a feeling of tugging and/or strain. It is often associated with a decrease in the cutaneous hydration rate and a modification of the barrier function, measured by the insensitive water loss.

In an unexpected way, the inventors noted that microorganisms in particular the probiotic microorganisms could prove to be effective, particularly in the adult, for the treatment of sensitive skin, particularly associated with dry skin provided that they are associated with an effective amount of at least one unsaturated fatty acid.

The inventors thereby discovered that the topical administration of such a composition, i.e. by direct application to the skin, proved to be particularly effective.

According to a first aspect, the object of the present invention is a cosmetic and/or dermatological topical composition, of particular use for the prevention and/or treatment of sensitive and/or dry skin, comprising at least an effective amount of at least one microorganism, in particular a probiotic microorganism and/or a fraction or a metabolite thereof in combination with an effective amount of at least one polyunsaturated fatty acid and/or polyunsaturated fatty acid ester and/or a salt or derivative thereof in a physiologically-acceptable vehicle.

According to another of its aspects, the object of the invention is a cosmetic method comprising at least one application step to the skin of a topical composition comprising at least an effective amount of at least one microorganism, in particular a probiotic microorganism and/or a fraction or a metabolite thereof in combination with an effective amount of at least one unsaturated fatty acid, and/or unsaturated fatty acid ester and/or a salt and/or derivative thereof in a physiologically-acceptable vehicle.

Still according to another of its aspects, the object of the invention is the use of an effective amount of at least one microorganism, in particular a probiotic microorganism and/or a fraction or a metabolite thereof in combination with an effective amount of at least one unsaturated fatty acid, and/or unsaturated fatty acid ester and/or a salt or derivative thereof for manufacturing a cosmetic or dermatological composition intended to treat or prevent sensitive skin disorders, whether or not associated with dry skin.

The association according to the invention can be formulated in oral or topical compositions.

“Effective amount” means, according to the present invention, a sufficient amount to obtain the expected effect.

Sensitive and/or Dry Skin

As specified previously, a sensitive skin is different from an allergic skin. Its reactivity does not result from an immunologic process and generally only results in dysesthesic sensations.

For obvious reasons, the absence of visible signs makes the diagnosis of sensitive skin difficult. Generally, this diagnosis relies on the questioning of the patient. Moreover, this symptomotology has an interest to make it possible to differentiate the sensitive skin, whether or not associated with dry skin, from the contact irritation or allergy for which there are, on the other hand, visible inflammatory signs.

Consequently, the development of “sensitive skin” products required to have evaluation tools for the skin sensory reaction. Since their design, the first tools were inspired by the essential characteristic of sensitive skin, namely the presence of discomfort signs induced by a topical application. Thereby, the lactic acid “stinging test” was the first test suggested. It is carried out by noting the tingling sensations reported by a volunteer after application of a 10% lactic acid solution on the sides of the nose. The subjects reporting moderate or strong tingling sensations are called “stingers” and considered as being with sensitive skin. Because of this cutaneous sensitivity to the topical application of a product, these subjects are then selected to test products known as “sensitive skin products”. More recently, to specifically activate the peripheral nerve endings, involved in the discomfort and called nociceptors, recently identified as being involved in sensitive skin, new tests were proposed which precisely use other discomfort inductors, such as capsaicin.

This second type of test, described in the application EP 1,374,913, constitutes another tool, particularly useful for the diagnosis of sensitive skin.

According to the present invention, the sensitive skin covers irritable skin and intolerant skin.

An intolerant skin is a skin which reacts by heating, tugging, formications and/or red spots sensations to various factors such as the application of cosmetic or dermatological products or soap. In general, these signs are associated with an erythema and a hyper-seborrheic or acneic skin, and even dermatitis, with or without dartre.

An irritable skin is a skin which reacts by a pruritus, i.e. by itching or tingling, to various factors such as the environment, emotions, food, wind, friction, razor, hard water with a high limestone concentration, variations in temperature, moisture or wool.

Generally, these two types of skin can be associated with a cutaneous dryness with or without dartre, or with a skin which presents an erythema.

As specified previously, the cutaneous dryness is often associated with a decrease in the cutaneous hydration rate, evaluated by corneometry, as well as with a deterioration of the barrier function, measured by the insensitive water loss.

The dry skin essentially occurs by a tugging and/or strain sensation. This one is also rough to touch and appears covered with scales. When the skin is slightly dry, these scales are abundant but not very visible to the naked eye. They are less and less numerous but increasingly visible to the naked eye when this disorder worsens.

The origin of this cutaneous dryness can be of constitutional or acquired type.

In the case of acquired dry skin, the interference of external parameters such as exposure to chemical agents, difficult climatic conditions, sunbeams or even certain therapeutic treatments (retinoids, for example) is crucial. Under these external influences, the skin can then become momentarily and locally dry. That can concern any type of skin.

In the case of the constitutional dry skin, two categories can be distinguished: pathological skin and nonpathological skin.

The pathological constitutional dry skin is primarily represented by atopic dermatitis and ichthyosis. They are almost independent of the external conditions.

The atopic dermatitis is described as being associated with a deficit in the metabolism of the lipids of the stratum corneum and notably of the ceramides. This pathology appears in the form of a xerosis, more or less chronic, concerning a large surface of the body, associated with inflammatory and pruriginous thrusts by plates.

The ichthyosis are the pathologies characterized by a genetic deficit affecting the keratinization process at various stages. They are shown by a great exfoliation by plates.

In the case of the nonpathological constitutional dry skin, the severity of the dryness state can depend on the external factors already mentioned. This category of skin covers the senile skin (characterized by a general reduction in the cutaneous metabolism with age), the fragile skin (very sensitive to the external factors and often accompanied by erythema and rosacea) and the vulgar xerosis (of probable genetic origin and appearing first and foremost on the face, the limbs and the back of the hands).

The compositions, methods and uses according to the invention, thereby prove particularly effective for the prevention and/or treatment of the sensitive and/or dry skin and more particularly the skin known as reactive, irritable and/or intolerant, the acquired dry skin and/or the constitutional dry skin.

Microorganisms and Particularly Probiotic Microorganisms

The microorganisms appropriate for the invention are microorganisms which can be administered to the animal or the human without any risks.

In particular, at least one microorganism known as probiotic type is used in the present invention.

According to the present invention, “probiotic microorganism” means a living microorganism which, when it is consumed in an adequate amount, has a positive effect on the health of its host “Joint FAO/WHO Expert Consultation on Evaluation of Health and Nutritional Properties of Probiotic in Food Including Powder Milk with Live Lactic Acid Bacteria, Oct. 6, 2001”, and which can particularly improve the intestinal microbial balance.

According to an alternative of the invention, this microorganism is implemented in an isolated form, i.e. not mixed with one or more compound(s) likely to be associated with it in its original environment.

According to the invention, “metabolite” means any substance resulting from the metabolism of the microorganisms considered according to the invention, and also granted with an effectiveness for the treatment of the sensitive and/or dry skin.

According to the invention, “fraction” more particularly means a moiety of said microorganism granted with an effectiveness for the treatment of the sensitive and/or dry skin by analogy with said entire microorganism.

The microorganisms appropriate for the invention can notably be selected amongst Ascomycetes such as Saccharomyces, Yarrowia, Kluyveromyces, Torulaspora, Schizosaccharomyces pombe, Debaromyces, Candida, Pichia, Aspergillus and Penicillium, bacteria of the Bifidobacterium, Bacteroides, Fusobacterium, Melissococcus, Propionibacterium, Enterococcus, Lactococcus, Staphylococcus, Peptostrepococcus, Bacillus, Pediococcus, Micrococcus, Leuconostoc, Weissella, Aerococcus, Oenococcus, Lactobacillus type, and mixtures thereof.

As Ascomycetes particularly appropriate for the present invention, Yarrowia lipolitica and Kluyveromyces lactis, can be mentioned in particular as well as Saccharomyces cereviseae, Torulaspora, Schizosaccharamyces pombe, Candida and Pichia.

Concerning the probiotic microorganisms, the following bacterial and yeast types are generally used:
Lactic bacteria: which produce lactic acid by fermentation of sugar. According to their morphologies, they are divided into two groups:
   Lactobacillus species: Lactobacillus acidophilus; amylovorus, casei, rhamnosus, brevis, crispatus, delbrueckii (subsp bulgaricus, lactis), fermentum, helveticus, gallinarum, gasseri johnsonii, paracasei, plantarum, reuteri, salivarius, alimentarius, curvatus, casei subsp. casei, sake
   Gocci: Enterococcus (faecalis, faecium), Lactococcus lactis (subspp lactis or cremoris), Leuconstoc mesenteroides subsp dextranicum, Pediococcus acidilactici, Sporolactobacillus inulinus, Streptococcus salvarius subsp. Thermophilus, Streptococcus thermophilus, Staphylococccus carnosus, Staphylococcus xylosus
Bifidobacteria or Bifidobacterium species: Bifidobacterium adolescentis, animalis, bifidum, breve, lactis, longum, infantis, pseudocatenulatum
Yeasts: Saccharomyces (cerevisiae or even boulardii),
Other spore-forming bacteria: Bacillus (cereus var toyo or subtilis), Bacillus coagulans, Bacillus licheniformis, Escherichia coli strain nissle, Propionibacterium freudenreichii,
and mixtures thereof.

The lactic bacteria and the bifidobacteries are the probiotics more frequently used.

Specific examples of probiotic microorganisms are Bifidobacterium adolescentis, Bifidobacterium animalis, Bifidobacterium bifidum, Bifidobacterium breve, Bifidobacterium lactis, Bifidobacterium longum, Bifidobacterium infantis, Bifidobacterium pseudocatenulatum, Lactobacillus acidophilus (NCFB 1748); Lactobacillus amylovorus, Lactobacillus casei (Shirota), Lactobacillus rhamnosus (GG strain), Lactobacillus brevis, Lactobacillus crispatus, Lactobacillus delbrueckii (subsp bulgaricus, lactis), Lactobacillus fermentum, Lactobacillus helveticus, Lactobacillus gallinarum, Lactobacillus gasseri, Lactobacillus johnsonii (CNCM I-1225), Lactobacillus paracasei, Lactobacillus plantarum, Lactobacillus reuteri, Lactobacillus salivarius, Lactobacillus alimentarius, Lactobacillus currvatus, Lactobacillus casei subsp. casei, Lactobacillus sake Lactococcus lactis, Enterococcus (faecalis, faecium), Lactococcus lactis (subsp lactis or cremoris), Leuconstoc mesenteroides subsp dextranicum, Pediococcus acidilactici, Sporolactobacillus inulinus, Streptococcus salvarius subsp. Thermophilus, Streptococcus thermophilus, Staphylococccus carnosus, Staphylococcus xylosus, Saccharomyces (cerevisiae or even boulardii), Bacillus (cereus var toyo or subtilis), Bacillus coagulans, Bacillus licheniformis, Escherichia coli strain nissle, Propionibactezium freudenreichii, and mixtures thereof.

The microorganisms can be formulated in powder state, i.e. in a dry form, or in the form of suspensions or solutions.

More particularly, they are probiotic microorganisms from the lactic bacteria group, notably like Lactobacillus and/or Bifidobacterium. As examples of these lactic bacteria, we can more particularly mention Lactobacillus johnsonii, Lactobacillus reuteri, Lactobacillus rhamnosus, Lactobacillus paracasei, Lactobacillus casei or Bifidobacterium bifidum, Bifidobacterium breve, Bifidobacterium longum, Bifidobacterium animalis, Bifidobacterium lactis, Bifidobacterium infantis, Bifidobacterium adolescentis or Bifidobacterium pseudocatenulatum, and mixtures thereof.

The particularly appropriate species are Lactobacillus johnsonii, Lactobacillus paracasei, Bifidobacterium adolescentis, Bifidobacterium longum and Bifidobacterium lactis NCC 2818 filed respectively according to the Treaty of Budapest with the Pasteur Institute (28 rue du Doctor Roux, F-75024 Paris cedex 15) on Jun. 30, 1992, Jan. 12, 1999, Apr. 15, 1999, Apr. 15, 1999, Jun. 7, 2005 under the following descriptions CNCM I-1225, CNCM I-2116, CNCM I-2168 and CNCM I-2170 and CNCM I-3446, and the Bifidobacterium longum (BB536) type and mixtures thereof.

According to a particular embodiment of the invention, the composition comprises at least two different microorganisms, particularly probiotic microorganisms and/or metabolites and/or fractions thereof. These microorganisms can differ in nature, for example bacterium and fungi, or even in family, in type, in species, or only in strain.

The composition according to the invention can thereby comprise at least one microorganism selected amongst those mentioned previously, and a second microorganism also selected amongst these microorganisms or not.

According to an alternative of the invention, the composition contains at least one Lactobacillus sp microorganism and at least one Bifidobacterium sp microorganism, notably in sufficient amounts to guarantee an administration at a rate of 10¹⁰ pfu/day, respectively.

The microorganisms and/or fractions and/or metabolites thereof can be formulated in a suitable vehicle in an amount equivalent to at least 10³ pfu/g, in particular at doses varying from 10⁵ to 10¹⁵ pfu/g, and more particularly from 10⁷ to 10¹² pfu/g of vehicle.

The compositions with topical application according to the invention generally comprise from 10³ to 10¹² pfu, in particular from 10⁵ to 10¹⁰ pfu, and more particularly from 10⁷ to 10⁹ pfu of microorganisms, in particular of probiotic microorganisms per gram of vehicle.

When the composition comprises metabolites, the metabolites contents in the compositions substantially correspond to the contents likely to be produced from 10³ to 10¹⁵ pfu, in particular from 10⁵ to 10¹⁵ pfu, and more particularly from 10⁷ to 10¹² pfu of living microorganisms per gram of vehicle.

The microorganism(s) can be included in the composition according to the invention in a living, semi-active or inactivated, dead form.

It/they can also be included in the form of fractions of cellular components or in the form of metabolites. The microorganism(s), metabolite(s) or fraction(s) can also be introduced in the form of a freeze-dried powder, a culture supernatant and/or in a concentrated form, if necessary.

According to a particular embodiment, the microorganisms are implemented in inactivated or even dead form, notably in the topical compositions.

Unsaturated Fatty Acids

According to the present invention, “unsaturated fatty acid” means a fatty acid comprising at least one double bond. They are more particularly fatty acids with long chains, i.e. being able to have more than 14 carbon atoms.

The unsaturated fatty acids can be in acid form, or in the form of salt, such as calcium salt thereof for example, or in the form of derivatives, notably of fatty acid ester(s).

The fatty acids can be monounsaturated such as petroselenic acid (in C₁₂), palmitoleic acid (in C₁₆) or oleic acid (in C₁₈), or can be polyunsaturated, i.e. presenting at least two double bonds.

It is understood that the selection of fatty acids is carried out by taking into account the finality of the composition which comprises them, i.e. intended for a topical application, or an oral administration or an airway administration.

“Airway” means the upper airways (such as the nasal cavity, the sinus, the mouth, the pharynx for example) and the pulmonary way.

The polyunsaturated fatty acids notably comprise ω-3 and ω-6 fatty acids, characterized by the closest unsaturation position to the terminal methyl group, and mixtures thereof.

Particularly appropriate for the invention are the unsaturated fatty acids comprising between 18 and 22 carbon atoms, in particular polyunsaturated fatty acids, notably ω-3 and ω-6 fatty acids.

Amongst the polyunsaturated fatty acids of the ω-6 series, we can mention in particular linolenic acid with 18 carbon atoms and two unsaturations (18:2, ω-6), γ-linolenic acid with 18 carbon atoms and three unsaturations (18:3, ω-6), dihomogamalinolenic acid with 20 carbon atoms and 3 unsaturations (20:3, ω-6), the arachidonic acid, 5,8,11,14 eicosatetraenoic acid (20:4, ω-6) and docosatetraenoic acid (22:4, ω-6).

The polyunsaturated fatty acids of the ω-3 series can notably be selected amongst α-linolenic acid (18:3, ω-3), stearidonic acid (18:4, ω-3), 5,8,11,14,17-eicosapentaenoic acid or EPA (20:5, ω-3), and 4,7,10,13,16,19-docosahexaenoic acid or DHA (22:6, ω-3), docosapentaenoic acid (22,5, ω-3), n-butyl-5,11,14-eicosatrienonic acid.

Particularly appropriate for the invention are the α-linolenic acid, γ-linolenic acid, stearidonic acid, eicosapentaenoic acid, docosahexaenoic acid, mixtures thereof or extracts comprising them.

According to an alternative of the invention, the fatty acid(s) considered is/are used in an isolated form, i.e. after extraction of its/their original source(s).

The fatty acid or fatty acid ester content, unsaturated or polyunsaturated in the compositions according to the invention can vary between 0.0001% and 90% in weight, notably between 0.01 and 50% in weight, and in particular between 0.1 and 10% in weight with respect to the total weight of the composition.

According to another alternative, the unsaturated fatty acids are added to the composition in the form of an oil or of a mixture of oils, rich in unsaturated fatty acids, i.e. whose unsaturated fatty acid content enables an effective amount of unsaturated fatty acids to be added, in particular of mono and/or polyunsaturated fatty acids.

These oils generally have an unsaturated fatty acid content higher than approximately 35%, in particular higher than or equal to 40% in weight, with respect to the total amount of fatty acids present in the oil.

According to an aspect of the invention, the oils considered as rich in polyunsaturated fatty acids will be used, i.e. whose polyunsaturated fatty acid content is higher than or equal to approximately 35%, even higher than or equal to approximately 40% with respect to the total amount of fatty acids present in the oil.

Preferably, the polyunsaturated fatty acids/monounsaturated fatty acids ratio in these oils is higher than 1, notably higher than or equal to 1.5.

The polyunsaturated fatty acid content can thereby be higher than or equal to 50%, even higher than or equal to 60%.

According to another aspect of the invention, oils rich in monounsaturated fatty acids are used, i.e. whose monounsaturated fatty acid content is higher than or equal to approximately 35%, even higher than or approximately equal to approximately 40% with respect to the total amount of fatty acids present in the oil.

Preferably, the monounsaturated fatty acids/polyunsaturated fatty acids ratio in these oils is higher than 1, notably higher than or equal to 1.5.

The monounsaturated fatty acid content can thereby be higher than or equal to 50%, even higher than or equal to 60%.

According to some embodiments of the invention, we can however use oils with an unsaturated fatty acid content lower than those defined above, but rich in certain specific unsaturated fatty acids.

By way of indication, we will use oils whose unsaturated fatty acid content of interest for example is higher than or equal to 15% in weight, with respect to the total amount of fatty acids into the oil composition.

The sources of γ-linolenic acid can be selected amongst vegetable oils such as evening primrose, borage, blackcurrant pips, echium and hemp oils, and spirulina algae extracts (Spirulina maxima and Spirulina platensis), for example.

The vegetable oils of nuts, hazelnuts, almonds (Juglans regia), coriander and soya (Glycina max), colza (Brassica naptus), chia, flax, muscat rose tree and fish oils, for example, are rich in ω-3 series polyunsaturated fatty acids.

The ω-3 polyunsaturated fatty acids can also be found in the zooplankton, shellfish/mollusks and fish. Fish oils constitute the main industrial source of EPA and DRA. The microalgae biomasses can also constitute a raw material for the extraction of ω-3 unsaturated fatty acids.

Thereby, the unsaturated fatty acid can be implemented in the composition in the form of at least one oil selected amongst oils such as evening primrose, borage, blackcurrant pips, nut, soya, fish, sunflower, wheat germ, hemp, fenugreek, muscat rose tree, echium, argan tree, baobab tree, rice bran, sesame, almond, hazelnut, chia, flax, olive, avocado, safflower, coriander and/or microalgae extract (for example spirulina), or zooplankton extract.

According to an embodiment of the invention, the unsaturated fatty acid can, in particular, be implemented in the form of at least one oil selected amongst evening primrose, borage, blackcurrant pips, nut, soya, fish, sunflower, wheat germ, hemp, fenugreek, muscat rose tree, echium, argan tree, baobab tree, rice bran, sesame, almond, chia, flax, safflower oils and/or microalgae extract (for example spirulina), or zooplankton extract.

Among these oils, we can use, for example, fish oil and sesame oil as a source of polyunsaturated fatty acids.

More particularly, we can select borage, safflower, hemp, chia (Salvia hispanica), echium, fenugreek, wheat germ, flax, nut, evening primrose, blackcurrant pips, muscat rose tree, soya or sunflower oils as a source of polyunsaturated fatty acids.

The oils particularly adapted to the contribution of monounsaturated fatty acids in the compositions according to the invention are notably selected amongst argan tree oil, rice bran oil, and in particular amongst almond oil, avocado oil, coriander oil, hazelnut oil and olive oil.

However, certain oils can be used at the same time as a source of mono- and/or poly-unsaturated fatty acids.

For this reason, we can mention baobab tree oil or rice bran oil for example, but also argan tree oil or sesame oil.

The compositions according to the invention can comprise these oils and/or extracts and/or biomasses in a content between 5 and 80% in weight, notably between 10 and 70% in weight with respect to the total weight of a composition, notably intended for an oral administration.

The compositions according to the invention can comprise these oils and/or extracts and/or biomasses at a concentration adjusted so that they are administered at a content between 0.1 g and 10 g/day, notably between 0.2 g and 5 g/day.

Of course, topical compositions, or associations according to the invention can further contain several other active agents.

As usable active agents, the B3, B5, B6, B8, C, E, or PP vitamins, the niacin, the carotenoids, the polyphenols and minerals such as zinc, calcium, magnesium etc. can be mentioned.

In particular, an antioxidant complex comprising C and E vitamins, and at least one carotenoid can be used, notably a carotenoid selected amongst β-carotene, lycopene, astaxanthin, zeaxanthin and lutein, flavonoids such as catechines, hesperidin, proanthocyanidins and anthocyanines.

It can also be at least one prebiotic or a mixture of prebiotics. More particularly, these prebiotics can be selected amongst oligosaccharides, produced from glucose, galactose, xylose, maltose, sucrose, lactose, starch, xylan, hemicellulose, inulin, acacia type gums for example, or a mixture thereof. More particularly, oligosaccharide comprises at least one fructo-oligosaccharide. More particularly, this prebiotic can comprise a mixture of fructo-oligosaccharide and inulin.

The compositions according to the invention can appear in all the galenic forms normally used, according to the administration mode chosen.

The vehicle can be of various natures according to the type of composition considered.

More particularly concerning the compositions for a topical administration, they can be aqueous, hydroalcoholic or oily solutions, solution-type dispersions or lotion or serum-type dispersions, emulsions having a liquid or semi-liquid consistency of milk type, suspensions or emulsions of cream type, aqueous or anhydrous gel, microemulsions, microcapsules, microparticles, or vesicular dispersions of ionic and/or nonionic type.

These compositions are prepared according to the usual methods.

These compositions can notably constitute cleansing, protection, treatment or care creams for the face, hands, feet, the large anatomical folds or the body, (for example day creams, night creams, make-up removers, basic foundation creams, anti-sun creams), make-up products like fluid foundations, make-up remover milks, body milks for protection or care, after-sun milks, lotions, gels or foams for skin care, like cleansing or disinfection lotions, anti-sun lotions, artificial suntan lotions, compositions for the bath, deodorant compositions containing a bactericidal agent, gels or after-shave lotions, depilatory creams, or compositions against insect bites.

The compositions according to the invention can also consist of solid preparations constituting soaps or cleansing bars.

They can also be used for the hair in the form of solutions, creams, gels, emulsions, foams or even in the form of aerosol compositions, also containing a propellant agent under pressure.

When the composition of the invention is an emulsion, the proportion of the fatty phase can be situated between 5 and 80% in weight, and preferably between 5 and 50% in weight with respect to the total weight of the composition. The oils, emulsifiers and coemulsifiers used in the composition in the form of emulsion are selected amongst those classically used in the cosmetic and/or dermatological field. The emulsifier and coemulsifier can be present, in the composition, in a proportion between 0.3 and 30% in weight, and preferably between 0.5 and 20% in weight with respect to the total weight of the composition.

When the composition of the invention is an oily solution or gel, the fatty phase can represent more than 90% of the total weight of the composition.

In a known way, the cosmetic and/or dermatological composition of the invention can also contain conventional additives in the cosmetic, pharmaceutical and/or dermatological field, such as hydrophilic or lipophilic gelling agents, lipophilic or hydrophilic active agents, preservative, antioxidants, solvents, fragrances, fillers, filters, bactericides, odor absorbers and dyes. The amounts of these various additives are those classically used in the field considered, and for example from 0.01 to 20% of the total weight of the composition. These additives, according to their nature, can be introduced in the fatty phase and/or the aqueous phase.

As fats usable in the invention, in addition to the unsaturated fatty acids, we can mention mineral oils such as hydrogenated polyisobutene and petroleum jelly oil for example, vegetable oils such as a liquid fraction of shea butter, sunflower and apricot almonds oil for example, animal oils such as perhydrosqualene, synthesis oils notably oil of Purcellin, isopropyl myristate and ethylhexyl palmitate for example, and fluorinated oils such as perfluoropolyethers for example. We can also use fatty alcohols, fatty acids such as stearic acid and waxes for example, notably of paraffin, carnauba and beeswax. We can also use silicone compounds like silicone oils, and cyclomethicone and dimethicone, waxes, resins and silicone gums for example.

As emulsifiers usable in the invention, we can mention glycerol stearate, polysorbate 60, the cetylstearylic alcohol/oxyethylenated cetylstearylic alcohol mixture with 33 mols of ethylene oxide for example, sold under the denomination Sinnowax AO® by the HENKEL company, the PEG-6/PEG-32/Glycol Stearate mixture sold under the denomination of Tefose® 63 by the GATTEFOSSE company, PPG-3 myristyl ether, silicone emulsifiers such as cetyldimethicone copolyol and sorbitan mono- or tristearate, PEG-40 stearate, oxyethylenated sorbitan monostearate (20OE).

As solvents usable in the invention, we can mention lower alcohols, notably ethanol and isopropanol, propylene glycol.

As hydrophilic gelling agents, we can mention carboxylic polymers such as carbomer, acrylic copolymers such as acrylate/alkylacrylate copolymers, polyacrylamides and notably the polyacrylamide C₁₃₋₁₄-Isoparaffin Laureth-7 mixture sold under the name of Sepigel 305® by the SEPPIC company, polysaccharides like cellulose derivatives, such as hydroxyalkylcelluloses and in particular hydroxypropylcellulose and hydroxyethylcellulose, natural gums such as guar, carob and xanthan, and clays.

As lipophilic gelling agents, we can mention modified clays such as the bentones, metal salts of fatty acids like aluminum stearates and hydrophobic silica, or ethylcellulose and polyethylene.

As hydrophilic active agents, we can use proteins or hydrolysates of protein, amino acids, polyols notably in C₂-C₁₀, such as glycerin, sorbitol, butylene glycol and polyethylene glycol, urea, allantoin, sugars and derivatives of sugar, water-soluble vitamins, starch, bacterial or vegetable extracts like those of Aloe Vera.

As lipophilic active agents, we can use retinol (vitamin A) and its derivatives, tocopherol (vitamin E) and its derivatives, ceramides, essential oils and unsaponifiables (tocotrienol, sesamin, gamma oryzanol, phytosterols, squalenes, waxes, terpenes).

Moreover, we can associate the active agents according to the invention, with active agents intended notably for the prevention and/or the treatment of skin problems.

The composition of the invention can also advantageously contain a thermal and/or mineral water, notably selected amongst Vittel water, waters of the Vichy basin and Roche Posay water.

The cosmetic treatment method of the invention can be notably implemented by applying the cosmetic and/or dermatological compositions or associations as defined above, according to the conventional technique of the use of these compositions. For example: applications of creams, gels, sera, lotions, make-up remover milks or after-sun compositions to the skin or dry hair, application of a hair lotion on wet hair, of shampoos, or application of toothpaste to the gums.

The cosmetic method according to the invention can be implemented by topical administration, a daily administration for example, of the association according to the invention which can for example be formulated in the form of gels, lotions, emulsions.

The method according to the invention can comprise a single administration. According to another embodiment, the administration is repeated 2 to 3 times daily for one day or more for example, and generally for an extended period of at least 4 weeks, even 4 to 15 weeks, with one or more periods of interruption if necessary.

The use according to the invention can be such that the compositions or associations defined above are implemented in a formulation intended for a topical use.

In the case of using an association according to the invention orally, using a vehicle ingestible is preferred.

Notably suitable as food or pharmaceutical vehicles are milk, yoghurt, cheese, fermented milks, milk-based fermented products, ice, products containing fermented cereals, milk-based powders, formulas for children and infants, confectionery food products, chocolate, cereals, animal food in particular domestic animals, pills, capsules or tablets, oral supplements in dry form and oral supplements in liquid form.

For ingestion, numerous embodiments of oral compositions, and notably of food product supplements, are possible. Their formulation is carried out using the usual methods to produce sugar-coated tablets, hard gelatin capsules, gels, emulsions, pills, capsules. In particular, the active agent(s) according to the invention can be incorporated in any other form of food product supplements or enriched food, for example food bars, or powders which are compacted or not. The powders can be diluted with water, in soda, dairy products or derived from soya, or be incorporated in food bars.

According to a particular embodiment, the microorganisms can be formulated within compositions in a encapsulated form so as to significantly improve their survival duration. In such a case, the presence of a capsule can in particular delay or avoid the degradation of the microorganism in the gastrointestinal tract.

If the microorganisms are formulated in a composition intended to be administered orally, this composition can comprise between 10³ and 10¹⁵ pfu/g of living microorganisms, in particular between 10⁵ and 10¹⁵ pfu/g, and more particularly between 10⁷ and 10¹² pfu/g of microorganisms per gram of vehicle, or at equivalent doses calculated for the inactive or dead microorganisms, or for microorganism fractions or metabolites produced.

In the particular case where the microorganism(s) is/are formulated in compositions administered orally, the microorganism(s) concentration, notably probiotic microorganism, can be adjusted so as to correspond to doses (expressed in microorganism equivalent) between 5.10⁵ and 10¹³ pfu/d, and in particular between 10⁷ and 10¹¹ pfu/d.

In the case of oral ingestion, the daily doses can, for ω-3 fatty acids, be situated between 0.5 and 2500 mg/d, notably between 5 and 500 mg/d, and for ω-6 fatty acids between 0.5 and 5000 mg/d, notably between 5 and 2000 mg/d.

In the case of using an association according to the invention by airway, the physiologically-acceptable vehicle is selected amongst those usually used by one skilled in the art to target the upper airways or the pulmonary way.

Thereby, the compositions intended for the upper airways can be presented, by way of example, in the shape of gargles, collutories, nasal preparations such as liquids for instillation or pulverization, powders or pomades.

The compositions intended to target the pulmonary way can be presented, notably, in the form of inhalation or aerosol.

Thereby, the compositions according to the invention can be formulated so as to be adapted to distribution by pulverizer.

The effective amount of microorganisms according to the invention to be implemented in the formulations for the airways are to be adapted according to the galenic form used and the targeted way.

For example, the effective amounts per gram of vehicle and/or to be administered per day can be as previously defined.

The preparation of the compositions intended to be administered by airway can be carried out in any way known by one skilled in the art.

In the description and the following examples, unless otherwise specified, the percentages are percentages in weight, and the ranges of values specified as “between . . . and . . . ” include the specified low and high limits. The ingredients are mixed, before their shaping, in the order and under conditions easily determined by one skilled in the art.

The examples hereinafter are presented by way of example, and do not limit the field of the invention.

### EXAMPLES OF COMPOSITIONS

### Example 1

### Face Lotion for Sensitive Skin

| | (% in weight) |
|---|---|
| Lactobacillus paracasei (CNCM I-2116) | 5.00 |
| Magnesium gluconate | 3.00 |
| Calcium Linoleate | 2.00 |
| Blackcurrant pips oil | 5.00 |
| Evening primrose oil | 2.00 |
| Borage oil | 5.00 |
| Antioxidant | 0.05 |
| Isopropanol | 40.00 |
| Preservative | 0.30 |
| Water qsp | 100.00 |

### Example 2

### Face Care Milk for Dry and Sensitive Skin

| | (% in weight) |
|---|---|
| Magnesium chloride | 3.00 |
| Calcium ascorbate | 3.00 |
| Lactobacillus paracasei (CNCM I-2116) | 5.00 |
| Bifido bacterium longum (CNCM I-2170) | 5.00 |
| Magnesium gluconate | 3.00 |
| Calcium Linoleate | 2.00 |
| Blackcurrant pips oil | 5.00 |
| Evening primrose oil | 2.00 |
| Borage oil | 5.00 |
| Antioxidant | 0.05 |
| Isopropanol | 20.00 |
| Glycerol stearate | 1.00 |
| Cetylstearylic alcohol/oxyethylenated | 3.00 |
| cetylstearylic alcohol with 33 mols OE | |
| (Sinnowax AO sold by the Henkel company) | |
| Cetylic alcohol | 1.00 |
| Dimethicone (DC 200 Fluid sold by the Dow | 1.00 |
| Corning company) | |
| Petroleum jelly oil | 6.00 |
| Isopropyl Myristate (Estol IPM 1514 sold | 3.00 |
| by Unichema) | |
| Antioxidant | 0.05 |
| Glycerin | 20.00 |
| Preservative | 0.30 |
| Water qsp | 100.00 |

### Example 3

### Face Care Gel for Sensitive Skin

| | (% in weight) |
|---|---|
| Lactobacillus johnsonii (CNCM I-1225) | 5.00 |
| Hydroxypropylcellulose (Klucel H sold by | 1.00 |
| the Hercules company) | |
| Bifido bacterium lactis (CNCM I-3446) | 5.00 |
| Magnesium gluconate | 3.00 |
| Calcium Linoleate | 2.00 |
| Blackcurrant pips oil | 5.00 |
| Evening primrose oil | 2.00 |
| Borage oil | 5.00 |
| Antioxidant | 0.05 |
| Vitamin C | 2.50 |
| Antioxidant | 0.05 |
| Isopropanol | 40.00 |
| Preservative | 0.30 |
| Water qsp | 100.00 |

### Example 4

### Face Care Milk for Dry and Sensitive Skin

| | (% in weight) |
|---|---|
| Magnesium ascorbate | 3.00 |
| Lactobacillus paracasei (CNCM I-2116) | 5.00 |
| Magnesium gluconate | 3.00 |
| Calcium Linoleate | 2.00 |
| Blackcurrant pips oil | 5.00 |
| Coriander oil | 2.00 |
| Borage oil | 5.00 |
| Antioxidant | 0.05 |
| Isopropanol | 40.00 |
| Preservative | 0.30 |
| Water qsp | 100.00 |

### Example 5

### Cream for the Care of Reactive Skin

| | (% in weight) |
|---|---|
| Bifidobacterium Longum (CNCM I-2170) | 5.00 |
| Coriander oil | 2.00 |
| Fish oil | 5.00 |
| Glycerol stearate | 1.00 |
| Cetylstearylic alcohol/oxyethylenated | 3.00 |
| cetylstearylic alcohol with 33 mols OE | |
| (Sinnowax AO sold by the Henkel company) | |
| Cetylic alcohol | 1.00 |
| Dimethicone (DC 200 Fluid sold by the Dow | 1.00 |
| Corning company) | |
| Petroleum jelly oil | 6.00 |
| Isopropyl Myristate (Estol IPM 1514 sold | 3.00 |
| by Unichema) | |
| Glycerin | 10.00 |
| Preservative | 0.30 |
| Water qsp | 100.00 |

### Example 6

### Unidose Powder Sachet (Orally)

| Active ingredient | mg/sachet |
|---|---|
| Lactobacillus lactis 12 (CNCM I-3446) | 100 |
| Magnesium citrate in magnesium mg | 300 |
| Calcium Citrate in calcium mg | 1000 |
| Fish oil | 100 |
| Borage oil | 100 |
| Nut oil | 100 |
| Excipient | |
| Maltodextrin | qsp |
| Sodium benzoate | qsp |

One to three sachets can be taken per day.

### Example 7

### Capsule

| | mg/capsule |
|---|---|
| Lactobacillus paracasei (CNCM I-2116) | 100.00 |
| Blackcurrant pips oil | 100.00 |
| Fish oil | 100.00 |
| Magnesium stearate | 0.02 |
| Natural flavor | qs |
| Excipient | qsp |

One to three of these capsules can be taken per day.

### Example 8

A vitamin complex is added to the formulation of example 7, which comprises 30 mg of vitamin C, 5 mg of vitamin E and 2 mg of lutein.

### Example 9

A vitamin complex is added to the formulation of example 7, which comprises 30 mg of vitamin C, 5 mg of vitamin E and 2 mg of lycopene by capsule.

### Example 10

### Capsule

| | mg/capsule |
|---|---|
| Vitamin C | 30 |
| Bifidobacterium longum (CNCM I-2170) | 50 |
| Lactobacillus paracasei (CNCM I-2116) | 50 |
| Blackcurrant pips oil | 100.00 |
| Excipients | qsp |
| Magnesium stearate | 0.02 |
| Natural flavor | qs |

One to three of these capsules can be taken per day.

### Example 11

A vitamin complex is added to the formulation of example 10, which comprises 5 mg of vitamin E and 2 mg of β-carotene or lutein.

### Example 12

A vitamin complex is added to the formulation of example 10, which comprises 5 mg of vitamin E and 2 mg of lycopene by capsule.

### Example 13

A mineral complex is added to the formulation of example 10, which comprises 200 mg of magnesium lactate and 400 mg of calcium lactate, 500 mg of polyphenol extracts.

### Example 14

### Double-Capsule

| | mg/capsule 1 | mg/capsule 2 |
|---|---|---|
| Lactobacillus paracasei (CNCM I-2116) | 50 | |
| Bifidobacterium longum (CNCM I-2170) | 50 | |
| Grap pips oil | | 150 |
| Coriander oil | | 150 |
| Vitamin E | | 5 |
| Carotenoid mixture | | 4 |
| Natural flavor | qs | qs |
| Excipient | qsp | qsp |

One to three capsules can be taken per day.

1.-22. (canceled)

23. A cosmetic and/or dermatological topical composition, notably useful for the prevention and/or treatment of sensitive and/or dry skin, comprising at least an effective amount of at least one probiotic microorganism and/or a fraction or a metabolite thereof in combination with an effective amount of at least one polyunsaturated fatty acid and/or polyunsaturated fatty acid ester and/or a salt or derivative thereof in a physiologically-acceptable vehicle.

24. A composition according to claim 23, wherein it comprises at least two different probiotic microorganisms, and/or fractions and/or metabolites thereof.

25. A composition according to claim 23, wherein said microorganism is selected from the group consisting of Ascomycetes such as Saccharomyces, Yarrowia, Kluyvero-myces, Torulaspora, Schizosaccharomyces pombe, Debaromyces, Candida, Pichia, Aspergillus and Penicillium, bacteria of the Bifidobacterium, Bacteroides, Fusobacterium, Melissococcus, Propionibacterium, Enterococcus, Lactococcus, Staphylococcus, Peptostrepococcus, Bacillus, Pediococcus, Micrococcus, Leuconostoc, Weissella, Aerococcus, Oenococcus, Lactobacillus type, and mixtures thereof.

26. A composition according to claim 23, wherein the microorganism is selected from the group consisting of the Saccharomyces cereviseae, Yarrowia lipolitica, Kluyveromyces lactis, Torulaspora, Schizosaccharomyces pombe, Candida, Pichia, Bifidobacterium bifidum, Bifidobacterium breve, Bifidobacterium longum, Bifidobacterium animalis, Bifidobacterium lactis, Bifidobacterium infantis, Bifidobacterium adolescentis, Bifidobacterium pseudocatenulatum, Lactobacillus acidophilus (NCF 748), Lactobacillus amylovorus, Lactobacillus casei (Shirota), Lactobacillus brevis, Lactobacillus crispatus, Lactobacillus fermentum, Lactobacillus helveticus, Lactobacillus gallinarum, Lactobacillus plantarum, Lactobacillus salivarius, Lactobacillus alimentarius, Lactobacillus casei subsp. casei, Lactobacillus paracasei, Lactobacillus curvatus, Lactobacillus delbruckii (subsp. bulgaricus lactis), Lactobacillus gasseri, Lactobacillus johnsonii, Lactobacillus reuteri, Lactobacillus rhamnosus (GG strain), Lactobacillus sake, Lactococcus lactis, Enterococcus (faecalis, faecium), Lactococcus lactis (subspp lactis or cremoris), Leuconstoc mesenteroides subsp dextranicum, Pediococcus acidilactici, Sporolactobacillus inulinus, Streptococcus salvarius subsp. Thermophilus, Streptococcus thermophilus, Staphylococccus carnosus, Staphylococcus xylosus Saccharomyces (cerevisiae or even boulardii), Bacillus (cereus var toyo or subtilis), Bacillus coagulans, Bacillus licheniformis, Escherichia coli strain nissle, Propionibacterium freudenreichii, and mixtures thereof.

27. A composition according to claim 23, wherein the microorganism originates from the lactic bacteria group.

28. A composition according to claim 23, wherein the microorganism is selected from the group consisting of Lactobacillus johnsonii (CNCM I-1225), Lactobacillus paracasei (CNCM I-2116), Bifidobacterium adolescentis (CNCM I-2168), Bifidobacterium longum (CNCM I-2170), Bifidobacterium lactis (CNCM I-3446), Bifidobacterium longum (BB536), and mixtures thereof.

29. A composition according to claim 23, wherein the probiotic microorganism and/or a fraction or a metabolite thereof are formulated in said vehicle in an amount equivalent to at least 10³ cfu/g of vehicle.

30. A composition according to claim 23, wherein the probiotic microorganism and/or a fraction or a metabolite thereof are formulated in said vehicle in an amount varying from 10³ to 10¹² cfu/g of vehicle.

31. A composition according to claim 23, wherein the probiotic microorganism and/or a fraction or a metabolite thereof are formulated in said vehicle in an amount varying from 10⁵ to 10¹⁰ cfu/g of vehicle.

32. A composition according to claim 23, wherein the polyunsaturated fatty acid is selected from the group consisting of w-3, w-6 polyunsaturated fatty acids, and mixtures thereof.

33. A composition according to claim 23, wherein the polyunsaturated fatty acid comprises between 18 and 22 carbon atoms.

34. A composition according to claim 23, wherein the polyunsaturated fatty acid is selected from the group consisting of linolenic acid, g-linolenic acid, dihomogamalinolenic acid, arachidonic acid, eicosatetraenoic acid, docosatetraenoic acid, a-linolenic acid, stearidonic acid, 5,8,11,14,17-eicosapentaenoic acid, 4,7,10,13,16,19-docosahexaenoic acid, docosapentaenoic acid, and n-butyl-5,11,14-eicosatrienonic acid.

35. A composition according to claim 23, wherein the polyunsaturated fatty acid is implemented in the form of at least one oil selected from the group consisting of evening primrose, borage, blackcurrant pips, nut, soya, fish, sunflower, wheat germ, hemp, fenugreek, muscat rose tree, echium, argan tree, baobab tree, rice bran, sesame, almond, chia, flax, safflower oils and/or microalgae extract (for example spirulina), or zooplankton extracts.

36. A composition according to claim 23, wherein the polyunsaturated fatty acid is present in a content between 0.0001 and 90% in weight, with respect to the total weight of the composition.

37. A composition according to claim 23, wherein the polyunsaturated fatty acid is present in a content between 0.01 and 50% in weight with respect to the total weight of the composition.

38. A composition according to claim 23, wherein the polyunsaturated fatty acid is present in a content between 0.1 and 10% in weight with respect to the total weight of the composition.

39. A composition according to claim 23, wherein it is presented in the form of aqueous, hydroalcoholic or oily solutions, of solution-type dispersions or lotion or serum-type dispensions, of emulsions having a liquid or semi-liquid consistency of the milk type, obtained by dispersion of a fatty phase in an aqueous phase (H/E) or conversely (E/H), or of suspensions or emulsions having soft, semi-solid or solid consistency of the cream type, of aqueous or anhydrous gel, or even of microemulsions, microcapsules, microparticles, or of vesicular dispersions of ionic and/or nonionic type.

40. A cosmetic method comprising at least one application step to the skin of a topical composition comprising at least an effective amount of at least one probiotic microorganism, and/or a fraction or a metabolite thereof in combination with an effective amount of at least one unsaturated fatty acid, and/or unsaturated fatty acid ester and/or a salt and/or derivative thereof in a physiologically-acceptable vehicle.

41. A method according to claim 40, wherein said microorganisms are at least two different probiotic ones.

42. A method according to claim 40, wherein the unsaturated fatty acid is a polyunsaturated fatty acid.

43. A method for manufacturing a cosmetic or dermatological composition intended to treat or prevent sensitive skin disorders whether or not associated with a dry skin, comprising a step of combining an effective amount of at least one probiotic microorganism and/or a fraction or a metabolite thereof with an effective amount of at least one unsaturated fatty acid, and/or unsaturated fatty acid ester and/or a salt and/or derivatives thereof.

44. A method according to claim 43, wherein the combination is formulated in a composition intended for a topical use.

45. A method according to claim 43, wherein the combination is formulated in a composition intended for an oral administration, or an airway administration.

46. A method according to claim 43, wherein said microorganisms are at least two different probiotic ones.

47. A method according to claim 43, wherein the unsaturated fatty acid is a polyunsaturated fatty acid.

48. A method according to claim 43, wherein the unsaturated fatty acid is a polyunsaturated fatty acid selected from the group consisting of w-3, w-6 polyunsaturated fatty acids, and mixtures thereof.
